(19) United States
(12) Patent Application Publication
(10) Pub. No.: US 2003/0165545 A1
(43) Pub. Date: Sep. 4, 2003

(54) Ophthalmic compositions including oil-in-water emulsions, and methods for making and using same

(75) Inventors: Stanley W. Huth, Newport Beach, CA (US); Orest Olejnik, Coto De Caza, CA (US); Zhi-Jian Yu, Irvine, CA (US)

Correspondence Address: STOUT, UXA, BUYAN & MULLINS LLP, 4 VENTURE, SUITE 300, IRVINE, CA, 92618, US

(73) Assignee: Allergan, Inc., Irvine, CA

(21) Appl. No.: 10/349,466

(22) Filed: Jan. 22, 2003

Related U.S. Application Data
(60) Provisional application No. 60/353,050, filed on Jan. 30, 2002.

(51) Int. Cl. A61K 9/00

(52) U.S. Cl. 424/400

(57) ABSTRACT

Ophthalmic compositions comprising an oil-in-water emulsion, preferably a self-emulsifying oil-in-water emulsion, including an oily component, an aqueous component and a surfactant component. The surfactant component may include three or more surfactants, for example, a polyoxyalkalene alkylene ether, a polyalkylene oxide ether of alkyl alcohol and a polyalkylene oxide ether of alkylphenol. The present compositions may include therapeutic components. Methods of making such compositions and using such compositions, for example, to treat eyes, to treat contact lenses and to provide desired therapeutic effects are provided.

### RELATED APPLICATION

This application claims the benefit of U.S. Provisional Application Serial No. 60/353,050 filed Jan. 30, 2002, the disclosure of which is incorporated in its entirety herein by reference.

### BACKGROUND OF THE INVENTION

The present invention relates to ophthalmic compositions and methods for making and using such compositions. More particularly, the invention relates to compositions comprising oil-in-water emulsions, preferably self-emulsifying oil-in-water emulsions, methods of making such compositions and methods of using such compositions.

Typical preparation of oil-in-water emulsions has involved dissolving water-soluble components in an aqueous phase and dissolving oil-soluble components in an oil phase. The oil phase is vigorously dispersion mixed into the aqueous phase, for example, at several thousand revolutions per minute (r.p.m.) for minutes to several hours. Manufacturing procedures employing such methods involve significant investment in capital equipment, are time consuming and cannot easily be scaled-up to larger batch sizes. Generally, it is difficult to stabilize oil-in-water emulsions prepared by these types of methodologies for a commercially desired shelf-life, for example, a shelf-life of about one year or two years or more, without incorporating viscosity builders to increase viscosity to relatively high levels. However, such relatively high viscosity is often undesirable for ophthalmic compositions and very often or even almost universally unacceptable for contact lens care compositions. A two-year shelf-life goal can sometimes be achieved if the emulsions are stored refrigerated. However, the use of refrigeration causes limitations for commercial distribution of the product.

Sterilization is essential for many oil-in-water emulsions which readily support the growth of bacteria giving rise to contamination of the composition. A problem encountered with emulsions produced by standard methods is that such emulsions are not easily sterilized using filtration techniques. Filter sterilization for ophthalmic compositions which comprise oil-in-water emulsions is preferred to heat sterilization because of problems associated with heat sterilization, such as manufacturing complexity, relatively high cost and the like. Also, precipitation and/or inactivation of composition components may occur in sterilization procedures where heat is used.

Additionally, oil-in-water emulsions with a low surfactant to oil ratio generally produce a higher degree of ocular comfort than those with a relatively high surfactant to oil ratio. Ocular comfort is of critical importance for commercial success in ophthalmic products such as contact lens multi-purpose compositions.

In view of these and other limitations to oil-in-water emulsions prepared by standard techniques, it would be advantageous to have ophthalmic compositions including oil-in-water emulsions which have one or two or more of the following advantageous properties: are easily prepared, are storage-stable, are easily sterilized, for example, using filter or filtration sterilization techniques, with little opportunity for microbial growth if contaminated, have a relatively low surfactant to oil ratio, have relatively low viscosity and are effective in performing the intended purpose or purposes of the composition.

### SUMMARY

Ophthalmic compositions comprising oil-in-water emulsions, preferably self-emulsifying oil-in-water emulsions, methods of preparing or making such compositions and methods of using such compositions have been discovered. The present emulsion-containing compositions are relatively easily and straight forwardly prepared and are storage-stable, for example, having a shelf life at about room temperature of at least about one year or about 2 years or more. In addition, the present compositions are advantageously easily sterilized, for example, using sterilizing filtration techniques, and eliminate, or at least substantially reduce, the opportunity or risk for microbial growth if the compositions become contaminated.

The present compositions preferably include self-emulsifying emulsions. That is, the present oil-in-water emulsions preferably can be formed with reduced amounts of dispersion mixing at shear speed, more preferably with substantially no dispersion mixing at shear speed. In other words, the present self-emulsifying emulsions preferably can be formed using reduced amounts of shear, and more preferably using substantially no shear. Further, the present emulsions have a relatively low weight ratio of emulsifying component or surfactant component to oil or oily component and, therefore, are advantageously safe and comfortable for topical ophthalmic application. Such oil-in-water emulsions, with a low surfactant to oil ratio, may be more readily prepared via self-emulsification than oil-in-water emulsions with a higher surfactant to oil ratio.

Topical ophthalmic application forms of the present compositions include, without limitation, eye drops for dry eye treatment and for other treatments, forms for the delivery of drugs or therapeutic components into the eye and forms for caring for contact lenses. The present compositions are very useful for treating dry eye and similar conditions, and other eye conditions. In addition, the present compositions are useful in or as carriers or vehicles for drug delivery, for example, a carrier or vehicle for delivery of therapeutic components into or through the eyes.

Contact lens care applications of the present compositions include, without limitation, compositions useful for cleaning, rinsing, disinfecting, storing, soaking, lubricating, re-wetting and otherwise treating contact lenses, including compositions which are effective in performing more than one of such functions, i.e., so called multi-purpose contact lens care compositions, other contact lens care-related compositions and the like. Contact lens care compositions including the present emulsions also include compositions which are administered to the eyes of contact lens wearers, for example, before during and/or after the wearing of contact lenses.

The integration of emulsions into contact lens care compositions, such as multi-purpose, re-wetting and other contact lens care compositions adds the additional utility or benefit of prevention of dry eye and provides lubrication to the lens and/or eye through mechanisms only emulsions can provide. Additional utilities or benefits provided by integrated emulsions in contact lens care compositions may include, without limitation, enhanced contact lens cleaning, prevention of contact lens water loss, inhibition of protein deposition on contact lenses and the like.

The present invention provides for ophthalmic compositions which include oil-in-water emulsions, preferably self-emulsifying oil-in-water emulsions. These oil-in-water emulsions comprise an oily component, for example, and without limitation, mineral oil; an aqueous component, which includes water; and a surfactant component which includes at least three emulsifiers or surfactants, for example, at least a first surfactant, a second surfactant and a third surfactant.

The oily component and the surfactant component or surfactants are advantageously chemically structurally compatible to facilitate self-emulsification of the emulsion.

In one embodiment, the surfactant component includes a first surfactant, a second surfactant and a third surfactant. Each of the surfactants is different, for example, in at least one aspect or feature or property, from the other surfactants. In a very useful embodiment, each surfactant includes a hydrophobic constituent and a hydrophilic constituent, with the hydrophobic constituent of the first surfactant and the hydrophobic constituent of the second surfactant being substantially similar, or even substantially identical, in chemical structure. The hydrophilic constituent of the first surfactant need not be chemically substantially similar or substantially identical in chemical structure to the hydrophilic constituents of the other surfactants. Preferably, the hydrophilic constituent of the second surfactant and the hydrophilic constituent of the third surfactant are substantially similar, or even substantially identical, in chemical structure. The hydrophobic constituent of the third surfactant need not be substantially similar or substantially identical in chemical structure to the hydrophobic constituents of the other surfactants or the oily component.

In one useful embodiment, the average hydrophile-lipophile balance (HLB) of the combined surfactant components preferably substantially equals the HLB or average HLB of the oily component. The surfactants included in the present compositions may be, and preferably are, non-ionic, although anionic, cationic and amphoteric surfactants may be employed.

The hydrophobic constituent of the first surfactant and the hydrophobic constituent of the second surfactant may be substantially similar in overall length in fully extended conformation. Fully extended conformation refers to the maximum linear extended conformation of a carbon atom-containing chain, for example, including a hydrophobic constituent of a surfactant. Differences in length in fully extended conformation between two different carbon atom-containing chains are often expressed in terms of methylene groups.

The hydrophobic constituent of the first surfactant and the hydrophobic constituent of the second surfactant may be substantially similar to a hydrophobic constituent of the oily component. Further, the hydrophobic constituent of the third surfactant may be shorter in overall length in fully extended conformation than the hydrophobic constituents of the first and second surfactants by an equivalent length of about 3 to about 10 methylene groups.

Any suitable combination of surfactants may be employed or included in the present invention, provided such surfactants function as described herein, provide effective and useful ophthalmic compositions and do not have any substantial or significant detrimental effect on the contact lens being treated by the present compositions, on the wearers of such contact lenses or on the humans or animals to whom such compositions are administered.

In one embodiment, the first surfactant is, without limitation, a polyoxyalkylene alkylene ether. In one embodiment, the polyoxyalkylene alkylene ether is a polyoxyethylene alkylene ether. In another embodiment, the polyoxyalkylene alkylene ether is a mixture of polyoxyethylene alkylene ethers and polyoxypropylene alkylene ethers.

In one embodiment, the second surfactant includes, without limitation, a polyalkylene oxide ether of an alkyl alcohol. In one embodiment, the polyalkylene oxide ether of an alkyl alcohol is a polyethylene oxide ether of an alkyl alcohol. In another embodiment, the polyalkylene oxide ether of an alkyl alcohol is a mixture of polyethylene oxide ethers of an alkyl alcohol and polypropylene oxide ethers of an alkyl alcohol.

The third surfactant may include, for example and without limitation, a polyalkylene oxide ether of an alkylphenol. In one embodiment, the polyalkylene oxide ether of an alkylphenol is a polyethylene oxide ether of an alkylphenol. In another embodiment, the polyalkylene oxide ether of an alkylphenol is a mixture of polyethylene oxide ethers of an alkylphenol and polypropylene oxide ethers of an alkylphenol.

In a particularly useful embodiment, the first surfactant is a polyoxyethylene oleyl ether, the second surfactant is a polyethylene oxide ether of stearyl alcohol, and the third surfactant is a polyethylene oxide ether of nonylphenol.

The ophthalmic compositions comprise an oily component which may include, without limitation, mineral oil and the like.

In another broad aspect of the invention, ophthalmic compositions comprising a therapeutic component and an oil-in-water emulsion, as described elsewhere herein, are provided. Such oil-in-water emulsions have been found to be very effective, and even superior, in or as carrier or vehicle components for the delivery of therapeutic components to or through the eye. Any therapeutic component or combination of therapeutic components may be included in the present compositions provided that such therapeutic component or components are effective when included and administered in the present compositions and have no substantial or significant detrimental or unacceptable effect on the present oil-in-water emulsions and/or the other components in the present compositions.

The therapeutic component preferably is present in an amount effective in providing a therapeutic effect to a patient in response to the composition being administered to the eye of the patient. Therapeutic components which may be included in the present compositions include, without limitation, antibacterial substances, antihistaminics, decongestants, anti-inflammatories, non-steroid anti-inflammatory drugs (NSAIDs), miotics, anticholinergics, mydriatics, antiglaucoma drugs, antiparasitic drugs, anti-protozoal drugs, antiviral drugs, carbonic anhydrase inhibitors, anti-fungal drugs, anesthetic agents, ophthalmic diagnostic drugs, ophthalmic agents used as adjuncts in surgery, chelating agents, immunosuppressive agents, quinoxalines, quinoxaline derivatives, timolol, timolol derivatives, pilocarpine, pilocarpine derivatives and the like and mixtures thereof. The therapeutic component may be effective in the eye and/or in one or more parts (or systemically) of the body of the human or animal to whom the composition is administered.

The compositions may contain additional substances, together with, or in embodiments without, a therapeutic component. For example, the compositions may contain one or more buffer components in an amount effective to provide the compositions with a desired pH. Any suitable buffer may be employed. The buffer component may be selected so as not to produce a significant amount of chlorine dioxide or evolve significant amounts of gas, such as carbon dioxide. The buffer component may be inorganic. Alkali metal and alkaline earth metal buffer components are advantageously used in the present invention. For example, phosphate buffers may be used in accordance with the present invention.

Tonicity components may be included in the present compositions in an amount effective to provide the compositions with a desired tonicity. Any suitable tonicity component may be employed. Examples of tonicity components include, without limitation, sodium chloride, potassium chloride, calcium chloride, magnesium chloride, dextrose, glycerin, propylene glycol, mannitol, sorbitol and the like and combinations or mixtures thereof.

Viscosity inducing components may be included. Any suitable viscosity inducing component may be employed. Such viscosity inducing components include, without limitation, water soluble natural gums, cellulose-derived polymers and the like and mixtures thereof. Useful natural gums include, without limitation, guar gum, gum tragacanth and the like and mixtures thereof. The viscosity inducing component may be selected from cellulosic derivatives and mixtures thereof. Useful cellulosic viscosity inducing components include hydroxypropyl cellulose, hydroxypropylmethyl cellulose, carboxymethyl cellulose, methyl cellulose, hydroxyethyl cellulose and the like and mixtures thereof. The viscosity inducing component preferably is selected from cellulosic derivatives and mixtures thereof.

A very useful viscosity inducing component is hydroxypropylmethyl cellulose (HPMC).

Carbopol polymers may also be employed as a viscosity inducing component.

The viscosity inducing component may be used in an amount effective to increase the viscosity of the composition, preferably to a viscosity in the range of about 1.5 to about 30, or even as high as about 750, cps at 25° C., preferably as determined by USP test method No. 911 (USP 23, 1995). To achieve this range of viscosity increase, an amount of viscosity inducing component of about 0.01% to about 5% (w/v) preferably is employed, with amounts of about 0.05% to about 0.5% being more preferred.

The present compositions may contain one or more suitable disinfecting agents, for example, and without limitation, polyhexamethylene biguanide (PHMB) and the like.

Other non-ionic surfactants, such as poloxamer 237 and the like and mixtures thereof, which preferably do not make a substantial or significant contribution to the emulsification or self-emulsification of the emulsions of the present compositions, may also be employed in accordance with the present invention. Vitamins such as Vitamin E tocopheryl polyethylene glycol 1000 succinate, hereinafter Vitamin E TPGS, and the like may be included in the compositions.

Additionally, contact lens wetting agents, contact lens cleaning agents, anti-microbial agents and the like and mixtures thereof may be included in the present compositions.

The present invention provides for methods of using ophthalmic compositions, such as the present ophthalmic compositions described elsewhere herein.

In one embodiment, the present methods comprise administering a composition of the invention to an eye of a subject, for example, a human or an animal, in an amount and at conditions effective to provide at least one benefit to the eye. In this embodiment, the present methods can employ a composition at least one portion of which, for example, a therapeutic component and the like, is useful for treating a condition, for example, dry eye and/or one or more other conditions of the eye.

In a very useful embodiment, the present methods comprise contacting a contact lens with a composition of the present invention in an amount and at conditions effective to provide at least one benefit to the contact lens and/or the wearer of the contact lens. In this embodiment, the present composition is employed as at least a portion of a contact lens care composition.

When the present compositions include a therapeutic component, such compositions may be used in methods which comprise administering the composition to an eye of a subject, that is a human or animal, in an amount effective in providing a desired therapeutic effect to the subject. Such therapeutic effect may be an ophthalmic therapeutic effect and/or a therapeutic effect directed to one or more other parts of the subject's body or systemically to the subject's body. In this embodiment, the present oil-in-water emulsion is employed as at least a portion of a composition useful as a carrier or vehicle for the therapeutic component.

The present invention provides for methods for preparing ophthalmic compositions which include oil-in-water emulsions, for example, self-emulsifying oil-in-water emulsions, as described elsewhere herein. In one embodiment, the present methods for preparing a composition comprise heating an oily component to a temperature above the melting temperature for the oily component. A surfactant component, as described elsewhere herein, is combined with the melted oily component to produce an admixture. In one embodiment, the surfactant component is dissolved in the oily component, for example, the melted oily component. The admixture may then be combined with, for example, mixed into, an aqueous phase. In one embodiment, the aqueous phase is heated to a temperature above the melting temperature of the melted oily component. Heating the aqueous phase may be done before combining or mixing the admixture with or into the aqueous phase. Further, these methods may include one or more steps of adding additional components to a composition.

In one embodiment, compositions of the present invention may be sterilized. For example, the compositions may be sterilized by heat, such as by autoclaving. In a particularly useful embodiment, the present compositions are sterilized by filtering or filtration.

Any and all features described herein and combinations of such features are included within the scope of the invention provided that such features of any such combination are not mutually inconsistent.

These and other aspects and advantages of the present invention are apparent in the following detailed description and claims.

### DETAILED DESCRIPTION

The present invention is directed to oil-in-water emulsion-containing compositions, preferably self-emulsifying oil-in-water emulsion-containing compositions that can be used in ophthalmic applications. For example, the compositions of the present invention are useful for drug delivery to or through the eye, for eye drops to treat dry eye and other eye conditions and for caring for contact lenses. Further, the present compositions can be used as artificial tear compositions, eyewash compositions, and irrigating compositions, for example, irrigating compositions during ophthalmic procedures, surgeries and the like.

The compositions of the present invention include emulsions, preferably self-emulsifying emulsions, including an oily component, such as one or more oils, for example, and without limitation, mineral oil and/or one or more other conventional well known and/or commercially available oils suitable for use in the present invention; a surfactant component which includes three or more surfactants; and an aqueous component which includes an aqueous phase. In addition, a number of additional components may be included in the present compositions. The compositions of the present invention are substantially non-toxic and/or non-irritating and/or non-damaging to the eye and can provide a protective function for ocular cells and tissues. Thus, the present compositions preferably are ophthalmically acceptable.

One or more oils or oily substances are used to form the present compositions. Any suitable oil or oily substance or combinations of oils or oily substances may be employed provided such oils and/or oily substances are effective in the present compositions, and do not cause any substantial or significant detrimental effect to the human or animal to whom the composition is administered, or to the contact lens being treated, or the wearing of the treated contact lens, or to the wearer of the treated contact lens. The oily component may, for example, and without limitation, be a higher fatty acid glyceride, for example, castor oil, corn oil, sunflower oil and the like and mixtures thereof. The oily component may include one or more non-polar oils such as mineral oil, silicone oil and the like and mixtures thereof.

Three or more surfactants may be used to form a surfactant component in accordance with the present invention. For example, three, four, five or more surfactants may be used to form the surfactant component.

In one particularly useful embodiment, three surfactants are included in a surfactant component used in the present invention. The surfactants useful to form the surfactant component in the present invention advantageously are water-soluble when used alone or as a mixture. These surfactants are preferably non-ionic.

Advantageously, the surfactant component includes three surfactants, the first surfactant, the second surfactant and the third surfactant, where each of these surfactants has a hydrophobic constituent and a hydrophilic constituent. In one embodiment, the hydrophobic constituent of the first surfactant and the hydrophobic constituent of the second surfactant are substantially similar to each other and the hydrophilic constituent of the second surfactant and the hydrophilic constituent of the third surfactant are substantially similar to each other. Further, the hydrophobic constituent of the first surfactant and the second surfactant may be substantially similar to the oily component employed in accordance with the invention. In one embodiment, the substantial similarities between constituents are in chemical structure and overall length of the constituents in fully extended conformation.

In one embodiment, the hydrophobic constituent of the third surfactant is shorter than the hydrophobic constituents of the first and second surfactants in fully extended conformation by about 2 or about 3 to about 10 or about 13 methylene (—CH₃—) groups.

Without wishing to limit the invention to any particular theory of operation, it is believed the advantageous self-emulsification property of the emulsion of the present invention is based upon molecular self-assembly of structurally related oil and surfactant molecules. Therefore, the oily components and the surfactant component employed may be chemically compatible to facilitate self-emulsification.

In one embodiment, the first and second surfactants have hydrophilic constituents that may or may not be similar in chemical structure. The hydrophilic constituent of one of the first and second surfactants is advantageously similar in structure, including overall length, to a hydrophilic constituent of the third surfactant employed. This third surfactant may have a hydrophobic constituent that is not necessarily similar to the hydrophobic constituents of the first and second surfactants or to the oily component employed. In this embodiment, the third surfactant's hydrophobic constituent may be shorter than the hydrophobic constituents of the other surfactants by an equivalent length of about 2 or about 3 to about 10 or about 13 methylene groups, as measured when all constituents are in fully extended conformations. In one example, exemplifying these principles, the oil used is mineral oil, the first surfactant is Brij® 93 (polyoxyethylene (2) oleyl ether), sold by ICI Americas, Inc.; the second surfactant is Lipocol® S-10 (10-mole ethylene oxide ether of stearyl alcohol), sold by LIPO Chemicals, Inc.; and the third surfactant is Makon® 10 (10-mole ethylene oxide ether of nonylphenol), sold by Stepan Company.

The amount of surfactant component present varies over a wide range depending on a number of factors, for example, the other components in the composition and the like. Often the total amount of surfactant component is in the range of about 0.001% to about 0.5%, for example, about 0.01% to about 0.5%, (w/v) of the composition.

A first surfactant that may be used in accordance with the present invention is a polyoxyalkylene alkylene ether. In one embodiment, the polyoxyalkylene alkylene ether is a polyoxyethylene alkylene ether. In another embodiment, the polyoxyalkylene alkylene ether is a mixture of polyoxyethylene alkylene ethers and polyoxypropylene alkylene ethers.

The alkylene group of the alkylene ether of the polyoxyalkylene alkylene ether may be, for example, between about 6 and about 20 or about 30 carbon atoms in length. In another example, the alkylene group is between about 14 and about 26 carbon atoms in length. In still another example, the alkylene group includes about 18 carbons. In one particularly useful embodiment, the polyoxyalkylene alkylene ether is a polyoxyethylene oleyl ether. For example, the polyoxyalkylene alkyl ether may be a polyoxyethylene (2) oleyl ether.

A second surfactant that may be used in accordance with the present invention is a polyalkylene oxide ether of an alkyl alcohol. In one embodiment, the polyalkylene oxide ether of an alkyl alcohol is a polyethylene oxide ether of an alkyl alcohol. In another embodiment, the polyalkylene oxide ether of alkyl alcohol is a mixture of polyethylene oxide ethers of an alkyl alcohol and polypropylene oxide ethers of an alkyl alcohol.

The alkyl group of the alkyl alcohol of the polyalkylene oxide ether of an alkyl alcohol may be, for example, between about 6 and about 20 or about 30 carbon atoms in length. In another example, the alkyl group is between about 14 and about 26 carbon atoms in length. The alkyl group may include about 18 carbons. In one particularly useful embodiment, the polyalkylene oxide ether of an alkyl alcohol is a polyethylene oxide ether of stearyl alcohol. For example, the polyoxyalkylene alkyl ether of an alkyl alcohol may be a 10-mole ethylene oxide ether of stearyl alcohol.

A useful third surfactant includes, for example, a polyalkylene oxide ether of an alkylphenol. In one embodiment, the polyalkylene oxide ether of an alkylphenol is a polyethylene oxide ether of an alkylphenol. In another embodiment, the polyalkylene oxide ether of an alkylphenol is a mixture of polyethylene oxide ethers of an alkylphenol and polypropylene oxide ethers of an alkylphenol.

The alkyl group of alkylphenol of the polyalkylene oxide ether of an alkylphenol may include, for example, between about 3 or about 4 and about 20 carbon atoms. For example, the alkyl group of the alkylphenol of a polyethylene oxide ether of alkyl phenol may include between about 3 or about 4 and about 20 carbon atoms. In another example, the alkyl group comprises between about 5 and about 15 carbon atoms. In still another example, the alkyl group includes about 9 carbon atoms. In one particularly useful embodiment, the polyalkylene oxide ether of an alkylphenol is a polyethylene oxide ether of nonylphenol. For example, the polyalkylene oxide ether of an alkylphenol may be a 10-mole polyethylene oxide ether of nonylphenol.

In one embodiment of the present invention, the polyoxyalkylene alkyl ether is a polyoxyethylene (2) oleyl ether, the polyoxyethylene oxide ether of an alkyl alcohol is a 10-mole ethylene oxide ether of stearyl alcohol, and the polyalkylene oxide ether of an alkylphenol is a 10-mole ethylene oxide ether of nonylphenol.

The ratio, for example, weight ratio, of the surfactant component to the oily component in the present oil-in-water emulsions is selected to provide acceptable emulsion stability and performance, and preferably to provide a self-emulsifying oil-in-water emulsion. Of course, the ratio of surfactant component to oily component varies depending on the specific surfactants and oil or oils employed, on the specific stability and performance properties desired for the final oil-in-water emulsion, on the specific application or use of the final oil-in-water emulsion and the like factors. For example, the weight ratio of the surfactant component to the oily component may range from about 0.05 or less to about 0.7 or more. Very useful oil-in-water emulsions in accordance with the present invention have surfactant component to oily component weight ratios in a range of about 0.1 to about 0.4 or about 0.5.

In one embodiment of the present invention, the compositions have a surfactant component to oily component weight ratio of about 0.217:1. These compositions may comprise, for example, 2.0 gm Brij® 93 (polyoxyethylene (2) oleyl ether); 15.0 gm of mineral oil; 0.50 gm of Lipocol® S-10 (10-mole ethylene oxide ether of stearyl alcohol); 0.75 gm Makon® 10 (10-mole ethylene oxide ether of nonylphenol); and 78.0 gm of a aqueous phase. In another embodiment, the compositions have a surfactant component to oily component weight ratio that is about 0.30:1. These compositions may comprise, for example, 15.0 gm of mineral oil; 2.0 gm Brij® 93 (polyoxyethylene (2) oleyl ether); 1.0 gm of Lipocol® S-10 (10-mole ethylene oxide ether of stearyl alcohol); 1.5 gm Makon® 10 (10-mole ethylene oxide ether of nonylphenol); and 78.0 gm of an aqueous phase.

Additionally, the average hydrophile-lipophile balance (HLB) of the combined surfactant component may advantageously be about equal to the HLB or average HLB emulsion requirement of the oil or oils used in the present compositions.

Poloxamer surfactants, which are polyoxyethylene, polyoxypropylene block polymers and the like, and are available from BASF Wyandotte Corp., Parsippany, N.J. 07054 under the trademark “Pluronic”, may also be employed. One such surfactant is Pluronic® F87, and is also known as poloxamer 237. Preferably, poloxamer surfactants as used herein do not contribute to the advantageous self-emulsification property of the present oil-in-water emulsions, but do contribute to the functional effectiveness, for example, cleaning, e.g., contact lens cleaning, effectiveness, of the present compositions.

The aqueous phase or component used in accordance with the present invention is selected to be effective in the present compositions and to have no substantial or significant deleterious effect, for example, on the compositions, on the use of the compositions, on the contact lens being treated, on the wearer of the treated lens, or on the human or animal in whose eye the present composition is placed.

The liquid aqueous medium or component of the present compositions preferably includes a buffer component which is present in an amount effective to maintain the pH of the medium or aqueous component in the desired range. The present compositions preferably include an effective amount of a tonicity adjusting component to provide the compositions with the desired tonicity.

The aqueous phase or component in the present compositions may have a pH which is compatible with the intended use, and is often in the range of about 4 to about 10. A variety of conventional buffers may be employed, such as phosphate, borate, citrate, acetate, histidine, tris, bis-tris and the like and mixtures thereof. Borate buffers include boric acid and its salts, such as sodium or potassium borate. Potassium tetraborate or potassium metaborate, which produce boric acid or a salt of boric acid in solution, may also be employed. Hydrated salts such as sodium borate decahydrate can also be used. Phosphate buffers include phosphoric acid and its salts; for example, M₂HPO₄ and MH₂PO4, wherein M is an alkali metal such as sodium and potassium. Hydrated salts can also be used. In one embodiment of the present invention, Na₂HPO₄.7H₂O and NaH₂PO₂.H₂O are used as buffers. The term phosphate also includes compounds that produce phosphoric acid or a salt of phosphoric acid in solution. Additionally, organic counter-ions for the above buffers may also be employed. The concentration of buffer generally varies from about 0.01 to 2.5 w/v % and more preferably varies from about 0.05 to about 0.5 w/v %.

The type and amount of buffer are selected so that the formulation meets the functional performance criteria of the composition, such as surfactant and shelf life stability, antimicrobial efficacy, buffer capacity and the like factors. The buffer is also selected to provide a pH, which is compatible with the eye and any contact lenses with which the composition is intended for use. Generally, a pH close to that of human tears, such as a pH of about 7.45, is very useful, although a wider pH range from about 6 to about 9, more preferably about 6.5 to about 8.5 and still more preferably about 6.8 to about 8.0 is also acceptable. In one embodiment, the present composition has a pH of about 7.0.

The osmolality of the present compositions may be adjusted with tonicity agents to a value which is compatible with the intended use of the compositions. For example, the osmolality of the composition may be adjusted to approximate the osmotic pressure of normal tear fluid, which is equivalent to about 0.9 w/v % of sodium chloride in water. Examples of suitable tonicity adjusting agents include, without limitation, sodium, potassium, calcium and magnesium chloride; dextrose; glycerin; propylene glycol; mannitol; sorbitol and the like and mixtures thereof. In one embodiment, a combination of sodium chloride and potassium chloride are used to adjust the tonicity of the composition.

Tonicity agents are typically used in amounts ranging from about 0.001 to 2.5 w/v %. These amounts have been found to be useful in providing sufficient tonicity for maintaining ocular tissue integrity. Preferably, the tonicity agent(s) will be employed in an amount to provide a final osmotic value of 150 to 450 mOsm/kg, more preferably between about 250 to about 350 mOsm/kg and most preferably between about 270 to about 320 mOsm/kg. The aqueous component of the present compositions more preferably is substantially isotonic or hypotonic (for example, slightly hypotonic, e.g., about 230 mOsm/kg) and/or is ophthalmically acceptable. In one embodiment, the compositions contain about 0.14 w/v % potassium chloride and 0.006 w/v % each of calcium and/or magnesium chloride.

In addition to tonicity and buffer components, the present compositions may include one or more other materials, for example, as described elsewhere herein, in amounts effective for the desired purpose, for example, to treat contact lenses and/or ocular tissues, for example, to provide a beneficial property or properties to contact lenses and/or ocular tissues, contacted with such compositions.

In one embodiment, the compositions of the present invention are useful, for example, as a carrier or vehicle, for the delivery of therapeutic agents to or through the eye. Any suitable therapeutic component may be included in the present compositions provided that such therapeutic component is compatible with the remainder of the composition, does not unduly interfere with the functioning and properties of the remainder of the composition, is effective, for example, to provide a desired therapeutic effect, when delivered in the present composition and is effective when administered to or through the eye. For example, in a very useful embodiment, the delivery of hydrophobic therapeutic components or drugs to or through the eye may be accomplished. Without wishing to limit the invention to any particular theory or mechanism of operation, it is believed that the oily component and the hydrophobic constituents of the surfactant components facilitate hydrophobic therapeutic components remaining stable and effective in the present compositions.

According to this aspect of the invention, an effective amount of a desired therapeutic agent or component preferably is physically combined or mixed with the other components of a composition of the present invention to form a therapeutic component-containing composition within the scope of the present invention.

The type of therapeutic agent or agents used will depend primarily on the therapeutic effect desired, for example, the disease or disorder or condition to be treated. These therapeutic agents or components include a broad array of drugs or substances currently, or prospectively, delivered to or through the eye in topical fashion or otherwise. Examples of useful therapeutic components include, but not limited to:

(1) antibacterial substances including quinolones, such as ofloxacin, ciprofloxacin, norfloxacin, gatifloxacin and the like; beta-lactam antibiotics, such as cefoxitin, n-formamidoyl-thienamycin, other thienamycin derivatives, tetracyclines, chloramphenicol, neomycin, carbenicillin, colistin, penicillin G, polymyxin B, vancomycin, cefazolin, cephaloridine, chibrorifamycin, gramicidin, bacitracin sulfonamides and the like; aminoglycoside antibiotics, such as gentamycin, kanamycin, amikacin, sisomicin, tobramycin and the like; naladixic acid and analogs thereof and the like; antimicrobial combinations, such as fluealanine/pentizidone and the like; nitrofurazones; and the like and mixtures thereof;

(2) antihistaminics and decongestants, such as pyrilamine, chlorpheniramine, phenylephrine hydrochloride, tetrahydrazoline hydrochloride, naphazoline hydrochloride, oxymetazoline, antazoline, and the like and mixtures thereof;

(3) anti-inflammatories, such as cortisone, hydrocortisone, hydrocortisone acetate, betamethasone, dexamethasone, dexamethasone sodium phosphate, prednisone, methylprednisolone, medrysone, fluorometholone, fluocortolone, prednisolone, prednisolone sodium phosphate, triamcinolone, indomethacin, sulindac, salts and corresponding sulfides thereof, and the like and mixtures thereof;

(4) non-steroid anti-inflammatory drug (NSAID) components, such as those which do or do not include a carboxylic (—COOH) group or moiety, or a carboxylic derived group or moiety; NSAID components which inhibit, either selectively or non-selectively, the cyclooxygenase enzyme, which has two (2) isoforms, referred to as COX-1 and COX-2; phenylalkoanoic acids, such as diclofenac, flurbiprofen, ketorolac, piroximcam and the like; indoles such as indomethacin and the like; diarylpyrazoles, such as celecoxib and the like; pyrrolo pyrroles; and other agents that inhibit prostaglandin synthesis and the like and mixtures thereof;

(5) miotics and anticholinergics, such as echothiophate, pilocarpine, physostigmine salicylate, diisopropylfluorophosphate, epinephrine, dipivolyl epinephrine, neostigmine, echothiopate iodide, demecarium bromide, carbachol, methacholine, bethanechol, and the like and mixtures thereof;

(6) mydriatics, such as atropine, homatropine, scopolamine, hydroxyamphetamine, ephedrine, cocaine, tropicamide, phenylephrine, cyclopentolate, oxyphenonium, eucatropine, and the like and mixtures thereof;

(7) antiglaucoma drugs, for example, adrenergic agonists such as quinoxalines and quinoxaline derivatives, such as (2-imidozolin-2-ylamino) quinoxaline, 5-halide-6-(2-imidozolin-2-ylamino) quinoxaline, for example, 5-bromo-6-(2-imidozolin-2-ylamino) quinoxaline and the like; timolol, especially as the maleate salt and R-timolol and a combination of timolol or R-timolol with pilocarpine and the like; epinephrine and epinephrine complex or prodrugs such as the bitartrate, borate, hydrochloride and dipivefrin derivatives and the like; hyperosmotic agents, such as glycerol, mannitol and urea and the like and mixtures thereof;

(8) antiparasitic compounds and/or anti-protozoal compounds, such as ivermectin; pyrimethamine, trisulfapyrimidine, clindamycin and corticosteroid preparations and the like and mixtures thereof;

(9) antiviral compounds, such as acyclovir, 5-iodo-2′-deoxyuridine (IDU), adenosine arabinoside (Ara-A), trifluorothymidine, interferon and interferon inducing agents, such as Poly I:C and the like and mixtures thereof;

(10) carbonic anhydrase inhibitors, such as acetazolamide, dichlorphenamide, 2-(p-hydroxyphenyl) thio-5-thiophenesulfonamide, 6-hydroxy-2-benzothiazolesulfonamide 6-pivaloyloxy-2-benzothiazolesulfonamide and the like and mixtures thereof;

(11) anti-fungal agents, such as amphotericin B, nystatin, flucytosine, natamycin, and miconazole and the like and mixtures thereof;

(12) anesthetic agents, such as etidocaine, cocaine, benoxinate, dibucaine hydrochloride, dyclonine hydrochloride, naepaine, phenacaine hydrochloride, piperocaine, proparadaine hydrochloride, tetracaine hydrochloride, hexylcaine, bupivacaine, lidocaine, mepivacaine and prilocaine and the like and mixtures thereof;

(13) ophthalmic diagnostic agents, such as

(a) those used to examine the retina, such as choride-sodium fluorescein and the like and mixtures thereof;

(b) those used to examine the conjunctiva, cornea and lacrimal structures, such as fluorescein and rose Bengal and the like and mixtures thereof; and

(c) those used to examine abnormal pupillary responses such as methacholine, cocaine, adrenaline, atropine, hydroxyamphetamine and pilocarpine and the like and mixtures thereof;

(14) ophthalmic agents used as adjuncts in surgery, such as alpha-chymotrypsin, and hyaluronidase and the like; visco-elastic agents, such as hyaluronates and the like and mixtures thereof;

(15) chelating agents, such as ethylenediamine tetraacetate (EDTA) and deferoxamine and the like; and mixtures thereof;

(16) immunosuppressive agents and anti-metabolites, such as methotrexate, cyclophosphamide, 6-mercaptopurine, cyclosporin and azathioprine and the like; and mixtures thereof;

(17) combinations of the above such as antibiotic/anti-inflammatory as in neomycin sulfate-dexamethasone sodium phosphate, quinolone-NSAID and the like; and concomitant anti-glaucoma therapy, such as timolol maleate-aceclidine and the like.

When a therapeutic component is present in the compositions of the present invention, the amount of such therapeutic component in the composition preferably is effective to provide the desired therapeutic effect to the human or animal to whom the composition is administered.

Typically, when a therapeutic component is present, the compositions comprising oil-in-water emulsions of the present invention contain from or at least about 0.001%, for example, about 0.01%, to about 5% (w/v) of the therapeutic component, e.g., medicament or pharmaceutical, on a weight to weight basis. Thus, for example, from one drop of a liquid composition which contains about 25 mg of composition, one would obtain about 0.0025 mg to about 1.25 mg of therapeutic component.

The particular therapeutic component, e.g., drug or medicament, used in the pharmaceutical compositions of this invention is the type which a patient would require or benefit from for the treatment, e.g., pharmacological treatment, of a condition which the patient has or is to be protected from or from which the patient is suffering. For example, if the patient is suffering from glaucoma, the drug of choice may be timolol and/or one or more other anti-glaucoma components.

It is within the knowledge of one skilled in the art to determine the correct amounts of therapeutic component, e.g., drug, to be added to a composition of the invention in order to assure the efficacious delivery of the desired therapeutic component.

Another aspect of this invention is the use of the herein described compositions comprising oil-in-water emulsions for the treatment of dry eye. For this use, one would administer a composition as needed as determined by one skilled in the art. For example, ophthalmic demulcents such as carboxymethylcellulose, other cellulose polymers, dextran 70, gelatin, glycerine, polyethylene glycols (e.g., PEG 300 and PEG 400), polysorbate 80, propylene glycol, polyvinyl alcohol, povidone and the like and mixtures thereof, may be used in the present ophthalmic compositions, for example, compositions useful for treating dry eye.

The demulcent components are present in such compositions, for example, in the form of eye drops, in an amount effective to reduce, or even substantially eliminate, the effects of dry eye in the human or animal into whose eye or eyes the composition is administered. The amount of demulcent component employed in the present compositions is similar to the amount of demulcent component used in commercially available eye drops used for treatment of dry eye. The amount of demulcent component present in the present compositions may be in a range of at least about 0.01% or about 0.05% to about 0.5% or about 1.0% (w/v) of the present composition.

In another embodiment, the present compositions are useful as multi-purpose care compositions, rewetting compositions and cleaning compositions, for example, in-the-eye cleaners, for contact lens care.

All types of contact lenses may be cared for using compositions of the present invention. For example, the contact lenses may be soft, rigid and soft or flexible gas permeable, silicone hydrogel, silicone non-hydrogel and conventional hard contact lenses.

A multi-purpose composition, as used herein, is useful for performing at least two functions, such as cleaning, rinsing, disinfecting, rewetting, lubricating, conditioning, soaking, storing and otherwise treating a contact lens, while the contact lens is out of the eye. Such multi-purpose compositions preferably are also useful for re-wetting and cleaning contact lenses while the lenses are in the eye. Products useful for re-wetting and cleaning contact lenses while the lenses are in the eye are often termed re-wetters or “in-the-eye” cleaners. The term “cleaning” as used herein includes the loosening and/or removal of deposits and other contaminants from a contact lens with or without digital manipulation and with or without an accessory device that agitates the composition. The term “re-wetting” as used herein refers to the addition of water over at least a part, for example, at least a substantial part, of at least the anterior surface of a contact lens.

Although the present compositions are very effective as multi-purpose contact lens care compositions, the present compositions, with suitable chemical make-ups, can be formulated to provide a single contact lens treatment. Such single treatment contact lens care compositions, as well as the multi-purpose contact lens care compositions are included within the scope of the present invention.

Methods for treating a contact lens using the herein described compositions are included within the scope of the invention. In general, such methods comprise contacting a contact lens with such a composition at conditions effective to provide the desired treatment to the contact lens.

The contacting temperature is preferred to be in the range of about 0° C. to about 100° C., and more preferably in the range of about 10° C. to about 60° C. and still more preferably in the range of about 15° C. to about 40° C. Contacting at or about ambient temperature is very convenient and useful. The contacting preferably occurs at or about atmospheric pressure. The contacting preferably occurs for a time in the range of about 1 minute or about 1 hour to about 12 hours or more.

The contact lens can be contacted with the composition, often in the form of a liquid aqueous medium, by immersing the lens in the composition. During at least a portion of the contacting, the composition containing the contact lens can be agitated, for example, by shaking the container containing the composition and contact lens, to at least facilitate the contact lens treatment, for example, the removal of deposit material from the lens. Before or after such contacting step, in contact lens cleaning, the contact lens may be manually rubbed to remove further deposit material from the lens. The cleaning method can also include rinsing the lens prior to the contacting step and/or rinsing the lens substantially free of the composition prior to returning the lens to a wearer's eye.

In addition, methods of applying or administering artificial tears, washing eyes and irrigating ocular tissue, for example, before, during and/or after surgical procedures, are included within the scope of the present invention. The present compositions, as described elsewhere herein, are useful as artificial tears, eyewash and irrigating compositions which can be used, for example, to replenish/supplement natural tear film, to wash, bath, flush or rinse the eye following exposure to a foreign entity, such as a chemical material or a foreign body or entity, or to irrigate ocular tissue subject to a surgical procedure. Foreign entities in this context include, without limitation, one or more of pollen, dust, ragweed and other foreign antigens, which cause adverse reactions, such as allergic reactions, redness, itching, burning, irritation, and the like in the eye.

The present compositions, having suitable chemical make-ups, are useful in each of these, and other, in-the-eye applications. These compositions can be used in in-the-eye applications in conventional and well-known manners. In other words, a composition in accordance with the present invention can be used in an in-the-eye application in a substantially similar way as a conventional composition is used in a similar application. One or more of the benefits of the present compositions, as discussed elsewhere herein, are provided as the result of such in-the-eye use.

A cleaning component may be included in the present compositions useful to clean contact lenses. When present, the cleaning component should be present in an amount effective to at least facilitate removing, and preferably effective to remove, debris or deposit material from a contact lens.

In one embodiment, cleaning enzymes are employed. A cleaning enzyme component can be provided in an amount effective to at least facilitate removing deposit material from the contact lens. Types of deposit material or debris which may be deposited on the lens include proteins, lipids, and carbohydrate-based or mucin-based debris. One or more types of debris may be present on a given lens.

The cleaning enzyme component employed may be selected from enzymes conventionally employed in the enzymatic cleaning of contact lenses. Among the preferred enzymes are proteases, lipases, and the like. Exemplary enzymes are described by Huth et al U.S. Pat. No. 32,672 RE and Karageozian et al U.S. Pat. No. 3,910,296, which disclosures are incorporated by reference herein.

Preferred proteolytic enzymes are those substantially free of sulfhydryl groups or disulfide bonds, the presence of which may react with active oxygen of an oxidative disinfectant, rendering the enzyme inactive. Metalloproteases, enzymes which contain a divalent metal ion, may also be used.

Yet a more preferred group of proteolytic enzymes are the serine proteases, such as those derived from Bacillus sp. and Streptomyces sp. bacteria and Aspergillus sp. molds. Of this class of enzymes, particularly useful enzymes are those derived from alkaline proteases, generically referred to as subtilisin enzymes.

Other enzymes for this application include pancreatin, trypsin, collaginase, keratinase, carboxylase, aminopeptidase, elastase, and aspergillopeptidase A and B, pronase E (from S. griseus) and dispase (from Bacillus polymyxa).

In one embodiment, a composition in accordance with the present invention containing such a cleaning enzyme component has sufficient enzyme to provide about 0.001 to about 3 Anson units of activity, for example, about 0.01 to about 1 Anson units, per single lens treatment. However, higher or lower amounts may be used. The preferred pH range for an enzyme can be determined by a skilled practitioner.

A particularly useful embodiment of the present compositions is one substantially free of proteolytic enzyme. Such a formulation, preferably with at least one additional surfactant, which advantageously does not substantially contribute to the self-emulsification property of the present oil-in-water emulsion, provides for effective contact lens cleaning without the need to rinse the lens after cleaning to free the lens of the enzyme, prior to placing the lens in the eye.

The present compositions may further comprise a disinfectant component. The amount of the disinfectant component present in the liquid aqueous medium is effective to disinfect a contact lens placed in contact with the composition.

When a disinfectant component is desired to be included in an instant composition, it may be an oxidative or a non-oxidative disinfectant component.

Particularly useful oxidative disinfectant components include hydrogen peroxide and/or one or more other peroxy-containing compounds, for example, one or more other peroxides, persalts and the like and mixtures thereof.

For hydrogen peroxide, a 0.5% (w/v) concentration, for example, in an aqueous liquid medium is often effective as a contact lens disinfectant component. It is preferred to use at least about 1.0% or about 2.0% (w/v) hydrogen peroxide which concentrations reduce the disinfecting time over that of the 0.5% (w/v) peroxide concentration. No upper limit is placed on the amount of hydrogen peroxide which can be used in this invention except as limited in that the disinfectant component should have no substantial detrimental effect on the contact lens being treated or on the eye of the wearer of the treated contact lens. An aqueous composition containing about 3% (w/v) hydrogen peroxide is very useful.

So far as other oxidative disinfectants, e.g., other peroxides, persalts and the like, are concerned, they should be used in effective disinfecting concentrations.

When an oxidative disinfectant is used in the present invention, a reducing or neutralizing component in an amount sufficient to chemically reduce or neutralize substantially all of an oxidative disinfectant, for example, hydrogen peroxide, present is employed.

Such reducing or neutralizing components are preferably incorporated into a tablet or like item. The reducing agent is generally any non-toxic reducing agent. Reducing components include, without limitation, SH (group)-containing water-soluble lower alcohols, organic amines and salts thereof, amino acids and di- or tripeptides, e.g., cysteine hydrochloride ethyl ester, gluthione, homocysteine, carbamoyl cysteine, cysteinylglycine, 2-mercaptopropionic acid, 2-mercaptopropionylglycine, 2-mercaptoethylamine hydrochloride, cysteine, n-acetylcysteine, beta mercaptoethanol, cysteine hydrochloride, dithiothreitol, dithioerythritol, sodium bisulfate, sodium metabisulfite, thio urea, sulfites, pyrosulfites and dithionites such as the alkali metal salts or alkaline earth metal salts of sulfurous acid, pyrosulfurous acid and dithionious acid, e.g., lithium, sodium, calcium and magnesium salts and mixtures thereof. The thiols, for example, N-acetylcysteine are particularly useful.

In general, the reducing component is used in amounts in the range of about 0.5% to about 10% (w/v) of the disinfectant-containing composition used.

In one embodiment, all or a portion of the reducing component is replaced by a catalase component which acts to catalyze the neutralization or decomposition of the oxidative disinfectant component, such as hydrogen peroxide. Such catalase component can be included, for example, in the core of a barrier component coated tablet, in an amount effective to, together with the reducing component, if any, destroy or cause the destruction of all the oxidative disinfectant component present in the disinfectant-containing composition used. Some excess catalase component may be advantageously used to increase the rate at which the oxidative disinfectant component is destroyed.

In one embodiment, for example, when a multi-purpose contact lens composition is desired, the disinfectant component is preferably a substantially non-oxidative disinfectant component. As used herein, non-oxidative disinfectant components include effectively non-oxidative organic chemicals which derive their antimicrobial activity through a chemical or physiochemical interaction with the microbes or microorganisms. Suitable non-oxidative disinfectant components are those generally employed in ophthalmic applications and include, but are not limited to, quaternary ammonium salts used in ophthalmic applications such as poly[dimethylimino-2-butene-1,4-diyl]chloride, alpha-[4-tris(2-hydroxyethyl) ammonium]-dichloride (chemical registry number 75345-27-6, available under the trademark Polyquaternium 1® from Onyx Corporation), benzalkonium halides, and biguanides such as salts of alexidine, alexidine-free base, salts of chlorhexidine, hexamethylene biguanides and their polymers, antimicrobial polypeptides, and the like and mixtures thereof. A particularly useful substantially non-oxidative disinfectant component is selected from one or more (mixtures) of tromethamine (2-amino-2-hydroxymethyl-1,3propanediol), polyhexamethylene biguanide (PHMB), N-alkyl-2-pyrrolidone, chlorhexidine, Polyquaternium-1, hexetidine, bronopol, alexidine, very low concentrations of peroxide, ophthalmically acceptable salts thereof, and the like and mixtures thereof.

The salts of alexidine and chlorhexidine can be either organic or inorganic and are typically disinfecting gluconates, nitrates, acetates, phosphates, sulphates, halides and the like. Generally, the hexamethylene biguanide polymers, also referred to as polyaminopropyl biguanide (PAPB), have molecular weights of up to about 100,000. Such compounds are known and are disclosed in U.S. Pat. No. 4,758,595 which is incorporated in its entirety by reference herein.

The non-oxidative disinfectant components useful in the present invention are preferably present in the present compositions in concentrations in the range of about 0.00001% to about 2% (w/v).

More preferably, the non-oxidative disinfectant component is present in the present compositions at an ophthalmically acceptable or safe concentration such that the user can remove the disinfected lens from the composition and thereafter directly place the lens in the eye for safe and comfortable wear.

When a contact lens is desired to be disinfected by a disinfectant component, an amount of disinfectant effective to disinfect the lens is used. Preferably, such an effective amount of the disinfectant reduces the microbial burden on the contact lens by one log order, in three hours. More preferably, an effective amount of the disinfectant reduces the microbial load by one log order in one hour.

The disinfectant component is preferably provided in the present composition, and is more preferably soluble in the aqueous component of the present composition.

The present compositions may include an effective amount of a preservative component. Any suitable preservative or combination of preservatives may be employed. Examples of suitable preservatives include, without limitation, benzalkonium chloride, methyl and ethyl parabens, hexetidine, phenyl mercuric salts and the like and mixtures thereof. The amounts of preservative components included in the present compositions are such to be effective in preserving the compositions and can vary based on the specific preservative component employed, the specific composition involved, the specific application involved, and the like factors. Preservative concentrations often are in the range of about 0.0000106 to about 0.05% or about 0.1% (w/v) of the composition, although other concentrations of certain preservatives may be employed.

Very useful examples of preservative components in the present invention include, but are not limited to, chlorite components. Specific examples of chlorite components useful as preservatives in accordance with the present invention include stabilized chlorine dioxide (SCD), metal chlorites such as alkali metal and alkaline earth metal chlorites, and the like and mixtures thereof. Technical grade (or USP grade) sodium chlorite is a very useful preservative component. The exact chemical composition of many chlorite components, for example, SCD, is not completely understood. The manufacture or production of certain chlorite components is described in McNicholas U.S. Pat. No. 3,278,447, which is incorporated in its entirety by reference herein. Specific examples of useful SCD products include that sold under the trademark Dura Klor by Rio Linda Chemical Company, Inc., and that sold under the trademark Anthium Dioxide® by International Dioxide, Inc. An especially useful SCD is a product sold under the trademark Purite® by Bio-Cide International, Inc.

Other useful preservatives include antimicrobial peptides. Among the antimicrobial peptides which may be employed include, without limitation, defensins, peptides related to defensins, cecropins, peptides related to cecropins, magainins and peptides related to magainins and other amino acid polymers with antibacterial, antifungal and/or antiviral activities. Mixtures of antimicrobial peptides or mixtures of antimicrobial peptides with other preservatives are also included within the scope of the present invention.

The compositions of the present invention may include viscosity modifying agents or components, such as cellulose polymers, including hydroxypropyl methyl cellulose (HPMC), hydroxyethyl cellulose (HEC), ethyl hydroxyethyl cellulose, hydroxypropyl cellulose, methyl cellulose and carboxymethyl cellulose; carbomers(e.g. carbopol. RTM); polyvinyl alcohol; polyvinyl pyrrolidone; alginates; carrageenans; and guar, karaya, agarose, locust bean, tragacanth and xanthan gums. Such viscosity modifying components are employed, if at all, in an amount effective to provide a desired viscosity to the present compositions. The concentration of such viscosity modifiers will typically vary between about 0.01 to about 5% w/v of the total composition, although other concentrations of certain viscosity modifying components may be employed.

It is desirable in some instances to include sequestering agents or components in the present compositions in order to, and in an amount effective to, bind metal ions, which, for example, might otherwise stabilize cell membranes of microorganisms and thus interfere with optimal disinfection activity. Alternatively, it is desirable in some instances to bind metal ions to prevent their interaction with other species in the compositions. Sequestering agents are included, if at all, in amounts effective to bind at least a portion, for example, at least a major portion of the metal ions present. Such sequestering components usually are present in amounts ranging from about 0.01 to about 0.2 w/v %. Examples of useful sequestering components include, without limitation ethylene-diaminetetraacetic acid (EDTA) and its potassium or sodium salts and low molecular weight organic acids such as citric and tartaric acids and their salts, e.g., sodium salts.

The present compositions may comprise effective amounts of one or more additional components. For example, one or more conditioning components; one or more contact lens wetting agents or one or more contact lens cleaning agents, for example, one or more vitamin or vitamin derivative components, for example, vitamin E TPGS (D-alpha-tocopheryl polyethylene glycol 1000 succinate); one or more stabilizers; one or more color indicators of hydrogen peroxide decomposition; one or more plasticizers; one or more wetting components; one or more wearability components, and the like and mixtures thereof may be included. Acceptable or effective concentrations for these and other additional components in the compositions of the invention are readily apparent to the skilled practitioner.

Each of the components may be present in either a solid or liquid form of the present compositions. When the additional component or components are present as a solid, they can either be intimately admixed such as in a powder or compressed tablet or they can be substantially separated, although in the same particles, as in an encapsulated pellet or tablet. The additional component or components can be in solid form until desired to be used, whereupon they can be dissolved or dispersed in the aqueous component of the present composition in order to, for example, effectively contact the surface of a contact lens.

When any component is included, it is preferably compatible under typical use and storage conditions with the other components of the composition.

In one example, preparation of the oil-in-water emulsions of the present invention is as follows. The two phases (oil and water) are preferably separately heated to an appropriate temperature. This temperature is the same in both cases, generally a few degrees to about 5° to about 10° C. above the melting point of the ingredient(s) having the highest melting point in the case of a solid or semi-solid oil or surfactant component (the three or more surfactants which contribute to the self-emulsification of the final emulsion) in the oil phase. Where the oil phase is liquid at room temperature, a suitable temperature for preparation of a composition may be determined by routine experimentation in which the melting point of the ingredients aside from the oily phase is determined in, for example, the oily phase or an aqueous phase. In cases wherein all components of either the oil phase or the water phase are soluble at room temperature, no heating may be necessary. Non-emulsifying agents which are water soluble components are dissolved in the water and oil-soluble components including the surfactant component are dissolved in the oil phase.

To create an oil-in-water emulsion, the final oil phase is gently mixed into either an intermediate, preferably de-ionized water, phase or into the final water phase to create a suitable dispersion and the product is allowed to cool with or without stirring. In the case wherein the final oil phase is first gently mixed into an intermediate water phase, the resulting emulsion concentrate is thereafter mixed in the appropriate ratio with the final aqueous phase. In such cases, the emulsion concentrate and the final aqueous phase may not be at the same temperature or heated above room temperature, as the emulsion may be already formed at this point.

The oil-in-water emulsions of the present invention can be sterilized after preparation using heat, for example, autoclave steam sterilization or can be sterile filtered using, for example, a 0.22 micron sterile filter. Sterilization employing a sterilization filter can be used when the emulsion droplet (or globule or particle) size and characteristics allows this. The droplet size distribution of the emulsion need not be entirely below the particle size cutoff of the 0.22 micron sterile filtration membrane to be sterile-filtratable. In cases wherein the droplet size distribution of the emulsion is above the particle size cutoff of the 0.22 micron sterile filtration membrane, the emulsion needs to be able to deform or change while passing through the filtration membrane and then reform after passing through. This property is easily determined by routine testing of emulsion droplet size distributions and percent of total oil in the compositions before and after filtration. Alternatively, a loss of a small amount of larger droplet sized material may be acceptable.

The present oil-in-water emulsions preferably are thermodynamicaly stable, much like microemulsions, and yet may not be isotropic transparent compositions as are microemulsions. The emulsions of the present invention advantageously have a shelf life exceeding one year at room temperature.

The following non-limiting examples illustrate certain aspects of the present invention.

### EXAMPLE 1

Shown are six contact lens multi-purpose compositions (MPS) with integrated oil-in-water emulsions of the present invention.

TABLE 1
MPS with integrated emulsions. All
concentrations are in w/v %

| Excipient | Formula 6 | Formula 7 | Formula 8 | Formula 9 | Formula 10 | Formula 11 |
|---|---|---|---|---|---|---|
| Na2HPO4.7 H2O | 0.12 | 0.12 | 0.12 | 0.12 | 0.12 | 0.12 |
| NaH2P04.H2O | 0.01 | 0.01 | 0.01 | 0.01 | 0.01 | 0.01 |
| NaCl | 0.69 | 0.69 | 0.69 | 0.69 | 0.69 | 0.69 |
| KCI | 0.14 | 0.14 | 0.14 | 0.14 | 0.14 | 0.14 |
| Glycerin | 0.2 | 0.2 | 0.2 | 0.2 | 0.2 | 0.2 |
| HPMC | 0.15 | 0.15 | 0.15 | 0.15 | 0.15 | 0.15 |
| Vit.E-TPGS | — | — | — | — | 0.06 | — |
| Pluronic F87 | — | — | — | 0.05 | — | — |
| Mineral oil | 0.5 | 1 | 2 | 1 | 1 | 0.9 |
| (Drakeol 10LT) | | | | | | |
| Mineral oil | — | — | — | — | — | 0.1 |
| (medium) | | | | | | |
| Lipcol S-10 | 0.0167 | 0.0333 | 0.0666 | 0.0333 | 0.0333 | 0.0333 |
| Brij93 | 0.0667 | 0.1333 | 0.2666 | 0.1333 | 0.1333 | 0.1333 |
| Mskon 10 | 0.025 | 0.05 | 0.1 | 0.05 | 0.05 | 0.05 |
| PHMB | 0.00011 | 0.00011 | 0.00011 | 0.00011 | 0.00011 | 0.00011 |
| Adjust pH if | 7.6 | 7.6 | 7.6 | 7.6 | 7.6 | 7.6 |
| necessary | | | | | | |

The compositions in Table 1 were prepared as follows: 30.0 gm of light mineral oil, NF grade (Drakeol® 10 LT from Penreco, Los Angeles, Calif.) was added to a 200 mL Erlenmeyer flask. A magnetic stir bar was added and the composition was heated to and maintained at 43° C. with gentle stirring. 1.00 gm of Lipocol® S-10 (LIPO Chemicals, Inc., Paterson, N.J.) was added and allowed to dissolve. Lipocol® S-10 is also known as Steareth-10, the 10-mole ethylene oxide ether of stearyl alcohol. It is non-ionic and is a solid at room temperature, with an HLB=12.42. The mineral oil composition at this point was slightly cloudy. 1.50 gm of Makon® 10 (Stepan Company, Northfield, Ill.) was added. Makon® 10 is the 10-mole ethylene oxide ether of nonylphenol, is non-ionic and is a liquid at room temperature. It has an HLB=13.33. 4.00 gm of Brij® 93 (Brij® 93 VEG from Uniqema, ICI Americas Inc, Wilmington, Del.) was added, whereupon the composition once again became clear. Brij® 93 is polyoxyethylene (2) oleyl ether, is non-ionic and is a liquid at room temperature. It has an HLB=4.94. The combined HLB of the three surfactants in this system is ((0.5×12.42)+(2.0×4.94)+(0.75×13.33))/(0.5×2.00+0.75)=8.02. Light mineral oil has an HLB requirement of about 10.

200 mL of deionized water was heated in a separate flask to 43° C. 158 mL of this were added to the flask containing the mineral oil and the three surfactants. The combined composition immediately self-emulsified to a homogeneous milky-white appearance without any stirring. The total composition volume of this emulsion concentrate is 200 mL.

Preparation of Final Emulsions:

The appropriate volume of the above emulsion concentrate was added at room temperature to a third flask containing water with the remaining dissolved components of each formulation, also at room temperature. For example, composition 6 in Table 1 was prepared by adding 66.7 mL of the emulsion concentrate to 1933.3 mL of aqueous solution containing all of the remaining ingredients of formula 6. Similarly, the remainder of the same emulsion concentrate, 133.3 mL, was added to 1866.7 mL of aqueous solution containing all of the remaining ingredients of formula 7 for that formula.

The final emulsion formulas were filter sterilized through a 0.22 micron cellulose acetate, low protein binding membrane (Corning Costar, Corning, N.Y.) into a sterile polystyrene flask for microbiology and other evaluations.

The total surfactant concentration of the emulsions in Table 1 ranges from 0.1084 w/v % for composition 6 to 0.2166 w/v % for composition 7 and 0.4332 w/v % for composition 8. The amount of surfactant required to emulsify 1.00 w/v % of mineral oil is 0.2166 w/v %. This is only 27% of the surfactant to oil ratio represented by the 1.00 w/v % amount of polysorbate 80 required to emulsify 1.25 w/v % castor oil in a preferred composition of ophthalmic oil-in-water emulsion which may be representative of a recently marketed oil-in-water ophthalmic emulsion for treatment of dry eye which requires conventional high-shear mixing during manufacture as disclosed in U.S. Pat. No. 5,981,607 which is incorporated in its entirety by reference herein.

The oil phase droplet size of formulas 6-11 in Table 1 was measured with a Beckman Coulter LS 230 Particle Size Analyzer immediately after manufacture and again after 8 and 10 months storage at room temperature in clear glass bottles. All formulas were very gently swirled for a few seconds prior to measurement. Table 2 presents the results.

TABLE 2
Oil droplet size of emulsion formulas, in
microns.

| | Initial | | | 8 months | |
|---|---|---|---|---|---|
| | (Sep. 23, 2000) | | | (May 29, 2001) | |
| Formula | Ave. Size | S.D. | Range | Ave Size | S.D. |
| 6 | 0.107 | 0.039 | .040-258 | 0.141 | 0.038 |
| 7 | 0.106 | 0.039 | .040.258 | 0.140 | 0.035 |
| 8 | 0.107 | 0.040 | .040-258 | 0.162 | 0.064 |
| 9 | 0.107 | 0.040 | .040.258 | 0.135 | 0.029 |
| 10 | 0.105 | 0.039 | .040-258 | 0.117 | 0.029 |
| 11 | 0.107 | 0.031 | .040.235 | 0.143 | 0.037 |

| | | | | |
|---|---|---|---|---|
| | 10 months | | | |
| | (Aug. 1, 2001) | | | |
| Formula | Ave Size | S.D. | Range | Observations |

| | | | | |
|---|---|---|---|---|
| 6 | 0.160 | 0.045 | .048-.342 | Slight creaming before |
| 7 | 0.164 | 0.054 | .040-.375 | swirling |
| 8 | 0.257 | 0.125 | .040-.598 | |
| 9 | 0.137 | 0.031 | .053-.258 | |
| 10 | 0.118 | 0.029 | .040-.235 | |
| 11 | 0.159 | 0.051 | .040-.375 | |

### EXAMPLE 2

Table 3 shows the antimicrobial activity of the emulsion formulas as prepared in Example 1. The table shows log reduction after 6 hours contact time. These initial test results are in parentheses and test results at 6 months are to the right of the parentheses. NR=no recovery=total kill. The initial inocula was 5-6 log for each organism.

TABLE 3
FDA soft contact lens disinfection panel of microorganisms

| Test Organism | Formula 6 | Formula 7 | Formula 8 | Formula 9 | Formula 10 | Formula 11 |
|---|---|---|---|---|---|---|
| S. marcescens | (NR) NR | (NR) NR | (NR) NR | (4.7) NR | (NR) NR | (3.7) NR |
| ATCC 13880 | | | | | | |
| S. aureus | (NR) NR | (NR) NR | (4.6) NR | (NR) NR | (NR) NR | (NR) NR |
| ATCC 6538 | | | | | | |
| P. aerugmosa | (NR) NR | (3.9) NR | (NR) NR | (NR) NR | (NR) NR | (NR) NR |
| ATCC 9027 | | | | | | |
| C. albicans | (1.0)2.7 | (1.0)2.7 | (1.5)2.2 | (1.2)2.5 | (1.0)2.0 | (1.5)2.5 |
| ATCC 10231 | | | | | | |
| F. solani | (1.5)1.7 | (1.6)1.7 | (1.5)1.8 | (1.2)1.7 | (1.6)1.8 | (1.5)1.7 |
| ATCC 36031 | | | | | | |

Note: the improvement seen for C. albicans observed for all formulas can be attributed to an improved test preparation procedure in effect at the 6 month time interval.
NR = no recovery

Table 4 shows the cytotoxicity of emulsion formulas as measured by neutral red retention.

TABLE 4

| Formula | 1 | 6 | 7 | 8 | 9 | 10 | 11 | 12 |
|---|---|---|---|---|---|---|---|---|
| % Neutral | 94.0 | 58.0 | 38.0 | 53.0 | 70.0 | 69.0 | 68.0 | 62.0 |
| Red- | | | | | | | | |
| Retention | | | | | | | | |
| @ 180 min | | | | | | | | |

Note: formula 1 is a non-emulsion formula, identical with formula 9 except no mineral oil, Lipocol

S-10, Brij® 93 and Makon® 10. Formula 12 is a marketed MPS (Complete®“B”), the formula of which is identical to formula 1 with the following exceptions: NaCl=0.79 w/v %, no glycerin, EDTA at 0.02 w/v % and pH=7.2.

The results presented in tables 1 through 4 indicate that the multi-purpose compositions with integrated emulsions are stable and substantially equivalent to non-emulsion multi-purpose compositions in terms of cytotoxicity and antimicrobial activity.

The emulsion formulas of Examples 1 and 2 have been shown to deposit a small amount of oil onto surfaces of soft contact lenses repeatedly soaked in the compositions. It is believed this layer of oil advantageously assists in preventing water loss, dehydration of soft contact lenses in the eye and protein deposition during contact lens wear. Without wishing to limit the invention to any theory or mechanism of operation, it is believed the oil layer prevents contact lens protein deposition during contact lens wear due to a shift in the critical surface energy of the surface towards values which make protein deposition less energetically favorable, akin to a Teflon®-coated surface.

### EXAMPLE 3

TABLE 5
SURFACTANT CONCENTRATION OPTIMIZATION EXPERIMENT

| | | | 3 | 4 | | |
|---|---|---|---|---|---|---|
| | 1 | 2 | Grams | average | 5 | |
| | Grams of | Grams | of | particle | Stand. | |
| | Lipocol | of Brij | Makon | size in | Dev. | |
| | S-10 | 93 | 10 | microns | (μm) | |
| 1 | 1.000 | 2.000 | 1.480 | .226 | .074 | |
| 2 | .500 | 1.000 | 0.740 | 38.000 | 18.350 | |
| 3 | .609 | 1.220 | 0.890 | 7.131 | 9.934 | |
| 4 | .694 | 1.420 | 1.050 | .889 | 1.260 | |
| 5 | .800 | 1.630 | 1.190 | .724 | 1.164 | |
| 6 | .520 | 1.990 | 0.730 | .165 | 0.038 | optimum |
| 7 | .523 | 2.040 | 1.490 | .211 | 0.064 | |
| 8 | .503 | 1.010 | 1.500 | 1000.000 | | |
| 9 | .200 | 2.000 | 1.480 | 1.121 | 1.319 | |
| 10 | .353 | 2.010 | 1.480 | .508 | 0.547 | |
| 11 | .503 | 2.030 | 0.730 | .173 | 0.042 | |
| 12 | .502 | 2.040 | 0.490 | 1000.000 | | |
| 13 | .498 | 1.800 | 1.480 | .564 | 0.693 | |
| 14 | .498 | 1.780 | 0.740 | .273 | 0.101 | |

Method of prep:
(1) Heat 15.0 gm Penreco Drakeol ® 10 LT mineral oil to 40-50° C.
(2) Add Lipocol S-10 and stir until it dissolves.
(3) Add Makon ® 10 and Brij ® 93.
(4) Heat DIH₂O 79.0 ml to 40-50° C., add to oil phase.
(5) Dilute emulsion from 5.0 ml to 100.0 ml with DIH₂O, measure oil globule size.
Note: The particle size measurements in table 5 are for relative comparisons only. This is because the average particle size as measured was later determined to be high due to a broken detector in the particle size analyzer (O.165 μm = 0.118 μm after the instrument was fixed).

### EXAMPLE 4

A contact lens is introduced into 1.8 mL of an emulsion of Example 1 (for example, Formula 6, 7, 8, 9, 10 or 11), which includes 0.0017 Anson Units of Subtilisin A. The Subtilisin A is effective to facilitate the removing, and preferably is effective to remove, debris or deposit material from the contact lens. Types of deposit material or debris which are deposited on the lens include proteins, lipids, and carbohydrate-based or mucin-based debris.

After at least 4 hours (or overnight) the cleaned contact lens is removed from the emulsion and placed directly into the eye for safe and comfortable wear. Alternatively, the cleaned and disinfected contact lens can be rinsed with, for example, conventional buffered saline or a composition of example 1 which does not include Subtilisin A before being placed in the eye for safe and comfortable wear.

### EXAMPLE 5

A contact lens is introduced into 2.0 mL of an emulsion of Example 1 (for example, Formula 6, 7, 8, 9, 10 or 11), which includes hydrogen peroxide, 0.5% (w/v) concentration. The hydrogen peroxide is effective to facilitate the disinfecting, and preferably is effective to disinfect the contact lens.

After soaking the contact lens overnight, the disinfected contact lens is removed from the composition and placed into a hydrogen peroxide neutralizing composition and, thereafter, placed into the eye for safe and comfortable wear.

### EXAMPLE 6

Shown below is the percent change with time of Intra Ocular Pressure (mm Hg) after an administration of a composition of Example 1 (for example, Formula 6, 7, 8, 9, 10 or 11) which includes about 0.131% 5-bromo-6-(2-imidozolin-2-ylamino) quinoxaline.

Approximately 0.05 mL of the composition is administered directly to the eye at time 0.

| 0 hr | administration of complex |
|---|---|
| 1 hr | −10.4% |
| 2 hr | −16.0% |
| 4 hr | −09.5% |
| 6 hr | −09.4% |

### EXAMPLE 7

A 34 year old female patient is diagnosed with dry eye syndrome. Approximately 0.05 mL of a composition of Example 1 (for example, Formula 6, 7, 8, 9, 10 or 11) is administered to the patient four times a day for two weeks. Administration of the composition is effective to treat the patient's dry eye condition.

The patient's symptoms which include general irritation and burning of the eyes disappear after the initial administration of the composition and do not reoccur before the following administration during the two week period.

While this invention has been described with respect to various examples and embodiments, it is to be understood that the invention is not limited thereto and that it can be practiced within the scope of the following claims.

What is claimed is:

1. An ophthalmic composition comprising an oil-in-water emulsion including an oily component, an aqueous component, and a surfactant component including a first surfactant, a second surfactant and a third surfactant, wherein each of the surfactants is different from the other surfactant.

2. The ophthalmic composition of claim 1 wherein the emulsion is a self-emulsifying emulsion.

3. The ophthalmic composition of claim 1 wherein each surfactant includes a hydrophobic constituent and a hydrophilic constituent, the hydrophobic constituent of the first surfactant and the hydrophobic constituent of the second surfactant are substantially similar in chemical structure, and the hydrophilic constituent of the second surfactant and the hydrophilic constituent of the third surfactant are substantially similar in chemical structure.

4. The ophthalmic composition of claim 1 wherein the hydrophobic constituent of the first surfactant and the hydrophobic constituent of the second surfactant are substantially similar in overall length in fully extended conformation.

5. The ophthalmic composition of claim 1 wherein the hydrophobic constituent of the first surfactant and the hydrophobic constituent of the second surfactant are substantially similar to a hydrophobic constituent of the oily component.

6. The ophthalmic composition of claim 1 wherein the hydrophobic constituent of the third surfactant is shorter in overall length in fully extended conformation than the hydrophobic constituents of the first and second surfactants by an equivalent length of about 3 to about 10 methylene groups.

7. The ophthalmic composition of claim 1 wherein the first surfactant is a polyoxyalkylene alkylene ether.

8. The ophthalmic composition of claim 1 wherein the second surfactant is a polyalkylene oxide ether of an alkyl alcohol.

9. The ophthalmic composition of claim 1 wherein the third surfactant is a polyalkylene oxide ether of an alkylphenol.

10. The ophthalmic composition of claim 1 wherein the first surfactant is a polyoxyethylene oleyl ether, the second surfactant is a polyethylene oxide ether of stearyl alcohol, and the third surfactant is a polyethylene oxide ether of nonylphenol.

11. The ophthalmic composition of claim 1 wherein the oily component comprises mineral oil.

12. The ophthalmic composition of claim 1 wherein the composition is sterilized by filtering.

13. An ophthalmic composition comprising an oil-in-water emulsion including an oily component, an aqueous component, and a surfactant component including a first surfactant, a second surfactant and a third surfactant, each surfactant includes a hydrophobic constituent and a hydrophilic constituent, the hydrophobic constituent of the first surfactant and the hydrophobic constituent of the second surfactant are substantially similar in chemical structure, and the hydrophilic constituent of the second surfactant and the hydrophilic constituent of the third surfactant are substantially similar in chemical structure.

14. The ophthalmic composition of claim 13 wherein the emulsion is a self-emulsifying emulsion.

15. The ophthalmic composition of claim 13 wherein the hydrophobic constituent of the first surfactant and the hydrophobic constituent of the second surfactant are substantially similar in overall length in fully extended conformation.

16. The ophthalmic composition of claim 13 wherein the hydrophobic constituent of the first surfactant and the hydrophobic constituent of the second surfactant are substantially similar to a hydrophobic constituent of the oily component.

17. The ophthalmic composition of claim 13 wherein the hydrophobic constituent of the third surfactant is shorter in overall length in fully extended conformation than the hydrophobic constituent of the first and second surfactants by an equivalent length of about 3 to about 10 methylene groups.

18. The ophthalmic composition of claim 13 wherein the first surfactant is a polyoxyalkylene alkylene ether.

19. The ophthalmic composition of claim 13 wherein the second surfactant is a polyalkylene oxide ether of an alkyl alcohol.

20. The ophthalmic composition of claim 13 wherein the third surfactant is a polyalkylene oxide ether of an alkylphenol.

21. The ophthalmic composition of claim 13 wherein the first surfactant is a polyoxyethylene oleyl ether, the second surfactant is a polyethylene oxide ether of stearyl alcohol, and the third surfactant is a polyethylene oxide ether of nonylphenol.

22. The ophthalmic composition of claim 13 wherein the oily component comprises mineral oil.

23. The ophthalmic composition of claim 13 wherein the composition is sterilized by filtering.

24. An ophthalmic composition comprising a therapeutic component and an oil-in-water emulsion including an oily component, an aqueous component and a surfactant component including a first surfactant, a second surfactant and a third surfactant, wherein each of the surfactants is different from the other surfactants.

25. The ophthalmic composition of claim 24 wherein the emulsion is a self-emulsifying emulsion.

26. The ophthalmic composition of claim 24 wherein the therapeutic component is present in an amount effective to provide a therapeutic effect to a patient in response to the composition being administered to an eye of the patient.

27. The ophthalmic composition of claim 24 wherein the therapeutic component is selected from the group consisting of antibacterial substances, antihistaminics, decongestants, anti-inflammatories, non-steroid anti-inflammatory drugs, miotics, anticholinergics, mydriatics, antiglaucoma drugs, antiparasitic drugs, anti-protozoal drugs, antiviral drugs, carbonic anhydrase inhibitors, anti-fungal drugs, anesthetic agents, ophthalmic diagnostic drugs, ophthalmic agents used as adjuncts in surgery, chelating agents, immunosuppressive agents and mixtures thereof.

28. The ophthalmic composition of claim 24 wherein the therapeutic component is selected from the group consisting of quinoxalines, quinoxaline derivatives, timolol, timolol derivatives, pilocarpine, pilocarpine derivatives and mixtures thereof.

29. The ophthalmic composition of claim 24 wherein each surfactant includes a hydrophobic constituent and a hydrophilic constituent, the hydrophobic constituent of the first surfactant and the hydrophobic constituent of the second surfactant are substantially similar in chemical structure and the hydrophilic constituent of the second surfactant and the hydrophilic constituent of the third surfactant are substantially similar in chemical structure.

30. The ophthalmic composition of claim 24 wherein the hydrophobic constituent of the first surfactant and the hydrophobic constituent of the second surfactant are substantially similar in overall length in fully extended conformation.

31. The ophthalmic composition of claim 24 wherein the hydrophobic constituent of the first surfactant and the hydrophobic constituent of the second surfactant are substantially similar to a hydrophobic constituent of the oily component.

32. The ophthalmic composition of claim 24 wherein the hydrophobic constituent of the third surfactant is shorter in overall length in fully extended conformation than the hydrophobic constituent of the first and second surfactants by an equivalent length of about 3 to about 10 methylene groups.

33. The ophthalmic composition of claim 24 wherein the first surfactant is a polyoxyalkylene alkylene ether.

34. The ophthalmic composition of claim 24 wherein the second surfactant is a polyalkylene oxide ether of an alkyl alcohol.

35. The ophthalmic composition of claim 24 wherein the third surfactant is a polyalkylene oxide ether of an alkylphenol.

36. The ophthalmic composition of claim 24 wherein the first surfactant is a polyoxyethylene oleyl ether, the second surfactant is a polyethylene oxide ether of stearyl alcohol, and the third surfactant is a polyethylene oxide ether of nonylphenol.

37. The ophthalmic composition of claim 24 wherein the oily component comprises mineral oil.

38. The ophthalmic composition of claim 24 wherein the composition is sterilized by filtering.

39. An ophthalmic composition comprising a therapeutic component, and an oil-in-water emulsion including an oily component, an aqueous component and a surfactant component including a first surfactant, a second surfactant and a third surfactant, each surfactant includes a hydrophobic constituent and a hydrophilic constituent, the hydrophobic constituent of the first surfactant and the hydrophobic constituent of the second surfactant are substantially similar in chemical structure, and the hydrophilic constituent of the second surfactant and the hydrophilic constituent of the third surfactant are substantially similar in chemical structure.

40. The ophthalmic composition of claim 39 wherein the emulsion is a self-emulsifying emulsion.

41. The ophthalmic composition of claim 39 wherein the therapeutic component is present in an amount effective to provide a therapeutic effect to a patient in response to the composition being administered to an eye of the patient.

42. The ophthalmic composition of claim 39 wherein the therapeutic component is selected from the group consisting of antibacterial substances, antihistaminics, decongestants, anti-inflammatories, non-steroid anti-inflammatory drugs, miotics, anticholinergics, mydriatics, antiglaucoma drugs, antiparasitic drugs, anti-protozoal drugs, antiviral drugs, carbonic anhydrase inhibitors, anti-fungal drugs, anesthetic agents, ophthalmic diagnostic drugs, ophthalmic agents used as adjuncts in surgery, chelating agents, immunosuppressive agents and mixtures thereof.

43. The ophthalmic composition of claim 39 wherein the therapeutic component is selected from the group consisting of quinoxalines, quinoxaline derivatives, timolol, timolol derivatives, pilocarpine, pilocarpine derivatives and mixtures thereof.

44. The ophthalmic composition of claim 39 wherein the hydrophobic constituent of the first surfactant and the hydrophobic constituent of the second surfactant are substantially similar in overall length in fully extended conformation.

45. The ophthalmic composition of claim 39 wherein the hydrophobic constituent of the first surfactant and the hydrophobic constituent of the second surfactant are substantially similar to a hydrophobic constituent of the oily component.

46. The ophthalmic composition of claim 39 wherein the hydrophobic constituent of the third surfactant is shorter in overall length in fully extended conformation than the hydrophobic constituent of the first and second surfactants by an equivalent length of about 3 to about 10 methylene groups.

47. The ophthalmic composition of claim 39 wherein the first surfactant is a polyoxyalkylene alkylene ether.

48. The ophthalmic composition of claim 39 wherein the second surfactant is a polyalkylene oxide ether of an alkyl alcohol.

49. The ophthalmic composition of claim 39 wherein the third surfactant is a polyalkylene oxide ether of an alkylphenol.

50. The ophthalmic composition of claim 39 wherein the first surfactant is a polyoxyethylene oleyl ether, the second surfactant is a polyethylene oxide ether of stearyl alcohol, and the third surfactant is a polyethylene oxide ether of nonylphenol.

51. The ophthalmic composition of claim 39 wherein the oily component comprises mineral oil.

52. The ophthalmic composition of claim 39 wherein the composition is sterilized by filtering.

53. A method of preparing an ophthalmic composition comprising:
providing an oily component to a temperature above a melting temperature of the oily component;
combining a surfactant component with the oily component to form an admixture, wherein the surfactant component includes a first surfactant, a second surfactant and a third surfactant, and each of the surfactants is different from the other surfactants; and
combining the admixture with an aqueous phase to form an oil-in-water emulsion.

54. The method of claim 53 wherein the oil-in-water emulsion formed is a self-emulsifying emulsion.

55. The method of claim 53 wherein each surfactant includes a hydrophobic constituent, and a hydrophilic constituent, the hydrophobic constituent of the first surfactant and the hydrophobic constituent of the second surfactant are substantially similar in chemical structure and the hydrophilic constituent of the second surfactant and the hydrophilic constituent of the third surfactant are substantially similar to each other.

56. The method of claim 53 wherein the hydrophobic constituent of the first surfactant and the hydrophobic constituent of the second surfactant are substantially similar in overall length in fully extended conformation.

57. The method of claim 53 wherein the hydrophobic constituent of the first surfactant and the hydrophobic constituent of the second surfactant are substantially similar to a hydrophobic constituent of the oily component.

58. The method of claim 53 wherein the hydrophobic constituent of the third surfactant is shorter in overall length in fully extended conformation than the hydrophobic constituent of the first and second surfactants by an equivalent length of about 3 to about 10 methylene groups.

59. The method of claim 53 wherein the combining of the surfactant component with the oily component is effective to dissolve the surfactant component in the oily phase.

60. The method of claim 53 wherein the first surfactant is a polyoxyalkylene alkylene ether, the second surfactant is a polyalkylene oxide ether of an alkyl alcohol, and the third surfactant is a polyalkylene oxide ether of an alkylphenol.

61. The method of claim 53 wherein the oily component comprises mineral oil.

62. The method of claim 53 further comprising sterilizing the oil-in-water emulsion by filtering the oil-in-water emulsion.

63. The method of claim 53 further comprising combining a therapeutic component with the oil-in-water emulsion.

64. A method comprising administering the composition of claim 1 to an eye of a subject in an amount effective to provide at least one benefit to the eye.

65. A method comprising administering the composition of claim 13 to an eye of a subject in an amount effective to provide at least one benefit to the eye.

66. A method comprising contacting a contact lens with the composition of claim 1 in an amount and at conditions effective to provide at least one benefit to the contact lens or to the wearer of the contact lens.

67. A method comprising contacting a contact lens with the composition of claim 13 in an amount and at conditions effective to provide at least one benefit to the contact lens or to the wearer of the contact lens.

68. A method comprising administering the composition of claim 24 to an eye of a subject in an amount effective in providing a desired therapeutic effect to the subject.

69. A method comprising administering the composition of claim 39 to an eye of a subject in an amount effective in providing a desired therapeutic effect to the subject.

70. A method comprising contacting a contact lens with a composition comprising an oil-in-water emulsion, the contacting being effective in providing the contact lens with at least one benefit selected from the group consisting of lubricating the contact lens, preventing dehydration of the contact lens and preventing protein deposition on the contact lens.
